(19) United States
(12) Patent Application Publication
(10) Pub. No.: US 2008/0208068 A1
(43) Pub. Date: Aug. 28, 2008

(54) DYNAMIC POSITIONAL INFORMATION CONSTRAINED HEART MODEL

(76) Inventors: Timothy Robertson, Belmont, CA (US); George M. Savage, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/037,851

(22) Filed: Feb. 26, 2008

Related U.S. Application Data
(60) Provisional application No. 60/891,683, filed on Feb. 26, 2007.
(60) Provisional application No. 60/893,545, filed on Mar. 7, 2007.

(51) Int. Cl. A61B 5/00; G06G 7/48

(52) U.S. Cl. 600/508; 703/11

(57) ABSTRACT

Methods and systems for producing a computational model of the heart which is patient-specific are provided. Embodiments of the methods and systems include providing a computational model of a heart, obtaining dynamic positional information data from a patient and modifying the heart model with the dynamic positional information data to produce a patient-specific heart model. The invention finds use in a variety of different applications, including but not limited to diagnosis and treatment of heart conditions.

### CROSS-REFERENCE TO RELATED APPLICATIONS

Pursuant to 35 U.S.C. § 119 (e), this application claims priority to U.S. Provisional Application Ser. No. 60/891,683 filed Feb. 26, 2007; and U.S. Provisional Application Ser. No. 60/893,545 filed Mar. 7, 2007; the disclosure of which priority applications is herein incorporated by reference.

### INTRODUCTION

Mathematical models can be extremely valuable in the diagnosis and treatment of certain ailments. Individual organs and biological systems can be modeled in silico, i.e. on a computer, in order to study them in situations that may not be feasible in vivo because of mechanical limitations, or undue risk to the patient. For example, several models of the heart are well known in the art, and can be used to study the effects of certain disorders and possible treatments.

Usyk and McCulloch teach a computational model of the dilated failing heart with left bundle branch block in “Electromechanical Model of Cardiac Resynchronization in the Dilated Failing Heart With Left Bundle Branch Block,” J Electrocardiol 2003; 36(suppl): 57-61, hereby incorporated by reference in its entirety. They proposed that the model they created was substantially predictive and could be a useful tool in determining optimal pacing conditions in a heart with these conditions.

The currently available models of the heart and other organs contain many free parameters that can vary among individuals. Parameters such as heart size, vascularization, and conduction channels, which can have a large effect on heart function, can vary dramatically from person to person.

### SUMMARY

The present invention provides a significant advancement in the art by using dynamic positional information data, optionally in conjunction with other data from other sources, to constrain a computational heart model, e.g., by removing or limiting one or more of the free parameters of the model, so as to produce a patient specific heart model that represents an individual patient's heart, such that the model is no longer generic but instead is specific for a patient. The inventive constrained patient-specific heart model allows for the first time a computational model of the heart or other organ or biological system to be individualized to a specific patient by fitting the model to data obtained through electric tomography. Using the patient specific model, data can be obtained that is not easily measured on the patient's actual heart.

Aspects of the invention include methods and systems for producing a computational model of the heart which is patient-specific are provided, i.e., a patient specific heart model. The invention finds use in a variety of different applications, including but not limited to diagnosis and treatment, predicting response to therapy, finding optimal pacing sites for cardiac resynchronization therapy (CRT), etc.

With the constrained heart model of the invention, the user can virtually perform therapies on the model before performing them on the patient. In some embodiments, one or more tests or therapies can be tested on the patient-specific heart model. The model can then generate predicted cardiac performance data as a result of the test or therapy. This feature allows the user, e.g., physician or other health care provider, to test several possible therapies, and determine which one will yield optimal results for the individual patient.

### BRIEF DESCRIPTION OF THE FIGURES

FIG. 1 shows a flow chart of an embodiment of a method of producing and using a constrained heart model of the invention

FIG. 2 shows a flow chart of an embodiment of a model refinement process of the invention.

### DETAILED DESCRIPTION

Methods and systems for producing a computational model of the heart which is patient-specific, i.e., a patient specific heart model, are provided. In methods of the invention, a heart model is modified with dynamic positional information data obtained from a patient to produce a patient specific heart model, which is referred to herein as a “constrained heart model.” In the constrained heart model, dynamic position information data, such as electric tomography (ET) data, and optionally data from other sources, is used to constrain a model of the heart, thereby individualizing the model for a specific patient.

Embodiments of the methods and systems include providing a computational model of a heart, and obtaining dynamic positional information data from a patient to modify the computational model of the heart. Modifying the computational heart model produces a patient-specific heart model, which model is predictive for the specific patient's heart function. In some embodiments, one or more additional types of data, i.e., parameters, is employed to modify the heart model to produce the patient specific heart model.

In further describing aspects of the invention, embodiments of methods of the invention are reviewed first in greater detail, followed by review of illustrative applications of the invention and a description of embodiments of systems and kits of the invention.

### Methods

As reviewed above, a first step of methods of the invention is to provide an initial computational model of a heart. In some embodiments, the initial computational model of the heart is a finite element model of the heart. In other embodiments, the initial computational model of the heart is one that is a numerical model prepared using finite difference methods or finite integration techniques. In certain embodiments, the initial computation model is one that has been prepared using boundary element methods. In certain embodiments, the initial model is a mathematical model, such as a series of equations, that predicts relevant data, e.g., ET or other data. In some embodiments, the initial model is one produced using FitzHugh-Nagumo equations. In some embodiments, impulse propagation can be modeled using a monodomain formulation.

There are multiple variables that can be included in a given initial heart model. Variables that may be present include, but are not limited to: cardiac geometry and anatomical divisions, muscle fiber orientation, the contractility in various places, the vascularization of the heart, properties of the conductive bundles, and the fluid dynamics of the blood. The complexity of the model can be chosen depending on how much data is available to fit the model to, and what data is desired to be studied in the model. Starting with a fairly simple model and fitting it to dynamic positional information data from a specific patient can yield a model which can reliably predict such data for other situations. The dynamic positional information data from the model can then be analyzed as it normally would be in vivo to determine heart performance. A more complex model can be used to create a more robust model that can yield a wide array of data on the performance of the heart as a result of various proposed treatments. A more complex model may require more data to be incorporated into it before the model becomes reliably predictive for the patient.

Examples of generic heart models that may be employed in the embodiments of the invention include, but are not limited to, those described in Sermesant et al., “Stimulation of cardiac pathologies using an electromechanical biventricular model and XMR interventional imaging,” Medical Image Analysis (2005) 9:467-480; Sermasant et al., “An electromechanical model of the heart for image analysis and simulation,” ISEE Transactions on Medical Imaging,” (2006) 25:612-625; Usyk et al., “Electromechanical Model of Cardiac Resynchronization in the Dilated Failing Heart with Left Bundle Branch Block,” J. Electrocardiology (supp 2003) 36: 57-61; U.S. Pat. No. 6,295,464 and U.S. Pat. No. 6,950,689; the disclosures of which are herein incorporated by reference.

The initial generic heart model may be obtained from any convenient source, including a commercial source, or prepared de novo. The initial generic heart model employed in the methods of the invention is characterized by not being specific for any particular patient.

As summarized above, following provision of the initial heart model, dynamic positional information data, such as electric tomography data (i.e., ET data), is obtained from a patient of interest. By “dynamic positional information” data is meant data which is obtained by detecting changes in an applied continuous field to obtain a signal, which signal is then employed to determine tissue location movement. Continuous field obtained data of interest includes electric tomography data as well as data obtained using other types of continuous fields, such as Doppler, magnetic, electromagnetic, pressure, light, etc. as described in U.S. patent application Ser. Nos. 11/664,340; 11/731,786; 11/909,786; 11562,690; 11/562,911; and 11/615,815; the disclosures of which are herein incorporated by reference. This type of dynamic positional information data is quantitative (and therefore not subjective) and may be referred to as quantitative real-time cardiac performance data. “Obtaining” data is used herein to refer to either determining dynamic positional information, e.g., ET, data in a patient or to the use of previously obtained dynamic positional information data for a patient. The source for the dynamic positional information data may therefore be a device configured to obtain patient-specific dynamic positional information data, or may be a device containing information on a patient's dynamic positional information data.

In certain embodiments, patient specific dynamic positional information data is ET data that is obtained as follows. Following implantation of any required elements in a subject (e.g., using known surgical techniques), the first step is to set up or produce, i.e., generate, a continuous electric field in a manner such that the tissue location(s) of interest is present in the generated continuous field. In certain embodiments, a single continuous field is generated, while in other embodiments a plurality of different continuous fields are generated, e.g., two or more, such as three or more, where in certain of these embodiments, the generated continuous fields may be substantially orthogonal to one another.

In practicing the subject methods, the applied continuous field may be applied using any convenient format, e.g., from outside the body, from an internal body site, or a combination thereof, so long as the tissue location(s) of interest resides in the applied continuous field. As such, in certain embodiments the applied continuous field is applied from an external body location, e.g., from a body surface location. In yet other embodiments, the continuous field is generated from an internal site, e.g., from an implanted device.

In certain embodiments, an electric tomography system is employed. In certain embodiments, the electric tomography system is one as described in U.S. patent application Ser. Nos. 11/664,340; 11/731,786; and 11/909/786; the disclosures of which are herein incorporated by reference. In electric tomography systems described in these applications, electric fields are generated in three dimensions such that the tissue of interest, such as the heart, is present in the electric fields. One or more leads, each with one or more electrodes, can be placed in the tissue to be studied such that each electrode is stably associated with a specific tissue location. By measuring aspects of the electric fields in each electrode, the location and motion of each electrode and the stably associated tissue location can be monitored. This data can then be used to determine many parameters of the heart, such as geometric information and dimensions of the heart, diastolic volume, systolic volume, ejection fraction, synchrony, and contractility.

Following obtainment of patient specific dynamic positional information data, the initial heart model is modified with the obtained data to produce the desired patient specific heart model, which patient specific heart model is predictive for said patient's heart function. The initial generic heart model may be modified with the patient specific dynamic positional information data using any convenient approach. In certain embodiments, the initial generic heart model is modified with the patient specific dynamic positional information data by employing model fitting techniques or protocols. In certain embodiments, model fitting techniques such as those discussed in “Optimal Control and Estimation,” by Robert F. Stengel (Dover Publications, 1994) are used to fit the model to the measured dynamic positional information data.

The generic heart model is modified with the patient specific dynamic positional information data, e.g., by employing model fitting techniques, until the desired patient specific heart model is obtained. In certain embodiments, the desired patient specific heart model is one that is predictive of heart function in the patient. In these embodiments, when the results of the model agree with measured results in the patient, a conclusion is made that the model is predictive for that particular patient.

Additionally, the patient-specific model of the heart can incorporate one or more additional non-dynamic positional information (i.e., non-DPI) parameters that can increase the reliability of the model. By non-DPI parameter is meant non-DPI data, as described below. These non-DPI parameters can include, but are not limited to, patient specific data regarding the geometry of the heart, the contractility in various places, the vascularization, properties of the conductive bundles, and the fluid dynamics of the blood, among other parameters.

In certain embodiments, several free parameters including dynamic positional information and non-DPI parameters can be adjusted until the results of the model agree with the measured results in the patient. By “free” parameter is meant any parameter that can be entered and/or modified, either by manual or automatic entering of data, such as automatically by software on a processor, into the initial generic model. In other embodiments of the constrained heart model, data from other sources can be combined with the electric tomography data to give a more robust model of the heart that incorporates more patient-specific parameters.

Non-EPI parameters of interest can vary greatly. Of interest in certain embodiments are direct parameters. For example, imaging data such as computed tomography (CT), magnetic resonance imaging (MRI) including MR tagging (e.g. MRI-SPAMM) and XMR (combined x-ray and MR systems), ultrasound (US), positron emission tomography (PET), and x-ray can be used to gather information on the geometry of the heart. Angiograms can be used to map out the coronary vessel structure, which varies greatly among different individuals. Angiographic data can then be incorporated into the model. A detailed map of the conduction pathways can be obtained through use of a catheter for electrophysiological (EPS) data mapping. IEGMs (intra cardiac electrograms) data can be obtained with the same leads used for ET measurements, and incorporated into the model. All of this data can be used as a direct parameter, i.e. a parameter directly used to constrain the computational heart model further. The more data that is incorporated into the model, the more accurate and robust the model will be.

In addition to directly measured parameters, there is also a large amount of other observed data with indirect effects that can be used to modify secondary structures of the patient-specific heart model. For example, the electric tomography system can give a velocity profile of a particular location in the heart. Other properties of the heart at that location, such as the geometry, conduction pathways, and vascularization can then be adjusted in an optimal model-fitting fashion until that location in the heart moves in the model as it was measured in the heart by DPI. In this way, a wealth of information can be used to indirectly constrain the free parameters in the heart model.

Another example of a measurement that can indirectly affect parameters in the heart model is the electrical delay time. For example, in patients with implanted devices this can be easily determined by measuring the electrical propagation time from when a pacing pulse is applied in the right atrium to when it is sensed in the left ventricle. This measurement does not give a direct map of the conduction structure of the heart, but can be used to optimize the conduction structure in the model.

Pressure-volume catheters can also yield many measurements which can be used by the model refinement system to constrain secondary parameters. Other examples of data that could be used to constrain the model either directly or indirectly include anatomic disruption of arterial wall, aneurysmal wall thickness, atherosclerotic plaque temperature, cerebral blood flow, cerebral blood volume, cerebral metabolic rate of oxygen (CMRO2), fibrous cap thickness, flow heterogeneity, increased pulmonary interstitial markings, intraluminal hemorrhage, intravascular thrombus, ischemic brain tissue, macrophage content, mean transit time, myocardial blood flow via 13N-ammonia, myocardial contractility/function, myocardial perfusion, neuronal activation, neuronal activation CMRO2, oxygen consumption, perfusion/diffusion, perianeurysmal fibrosis, splenic tissue characterization, subendothelial lipid pool, targeted microbubble contrast agents, vascular diameter and circumference, vascular lumen diameter, vascular occlusion, ventilation/perfusion mismatch, fibrilation state, activity state, heart rate, heart rate variability, fluid status and respiration rate.

Many of these parameters have been shown to correspond to a cardiac disease or condition. The disease or condition that each of the biomarkers corresponds to, the imaging technique(s) that can be used to detect the biomarker, and the investigator(s) that have demonstrated the correlation between the biomarker and the cardiac condition are shown in Table 1. In some embodiments the condition which is suggested by the biomarker can be incorporated into the heart model when the biomarker is detected.

TABLE 1

| | Disease/ | | |
|---|---|---|---|
| | Medical | | |
| Biomarker | Condition | Imaging Technique | Investigators |
| Anatomic disruption | Aortic dissection | US, CT, MRI, Angiography | Gonzalez R; Waltman A |
| of arterial wall | | | |
| Anatomic disruption | Carotid dissection | US, CT/CTA, MRI/MRA, | Gonzalez R; Waltman A |
| of arterial wall | | Angiography | |
| Anatomic disruption | Intracranial arterial | CTA, MRA, Angiography | Gonzalez R; Lev MH; |
| of arterial wall | dissection | | Waltman A |
| Aneurysmal wall | Inflammatory | CT | Brady T; Saini S; Waltman A |
| thickness | abdominal aortic | | |
| | aneurysm (IAAA) | | |
| Atherosclerotic | Vulnerable plaque | Thermography | Brady T; Lev M; Waltman A |
| plaque temperature | | | |
| Cerebral blood flow | Stroke | MRI (combined Arterial spin | Harris G; Hoge R; |
| (CBF) | | labeling and Dynamic | Ostergaard L; Rosen B; |
| | | susceptibility contrast), | Sorensen AG; Wald LL |
| | | CAPTIVE | |
| Cerebral blood flow | Stroke/ischemic | CAPTIVE, MRI | Bogdanov A; Jenkins B; |
| (CBF) | | | Kwong K; Weissleder R |
| Cerebral blood | Brain function/drugs | fMRI | Jenkins B; Kosofsky B; |
| volume (CBV) | of abuse | | Mandeville JB; Rosen B |
| Cerebral blood | Stroke | MRI (combined Arterial spin | Harris G; Hoge R; |
| volume (CBV) | | labeling and Dynamic | Ostergaard L; Rosen B; |
| | | susceptibility contrast), | Sorensen AG; Wald LL |
| | | CAPTIVE | |
| CMRO2 | Stroke | fMRI, Diffuse optical | Boas D; Mandeville JB; |
| | | topography (Near infrared | Strangman G |
| | | spectroscopy) | |
| Fibrous cap | Vulnerable plaque | Optical coherence topography | Weissleder R |
| thickness | | | |
| Flow heterogeneity | Stroke | MRI (combined Arterial spin | Harris G; Hoge R; |
| (FH) | | labeling and Dynamic | Ostergaard L; Rosen B; |
| | | susceptibility contrast) | Sorensen AG; Wald LL |
| | | CAPTIVE | |
| Increased pulmonary | Interstitial pulmonary | CT | McLoud T; Shepard J |
| interstitial markings | edema | | |
| Intraluminal | Gastrointestinal | SPECT | Fischman AJ |
| hemorrhage | bleeding | | |
| Intravascular | Acute myocardial | MRI | Brady T |
| thrombus | infarction | | |
| Intravascular | Arterial embolism | CT, MRI, Angiography | Waltman A |
| thrombus | | | |
| Intravascular | Deep venous | US, MRI, Angiography | O'Neill MJ; Waltman A |
| thrombus | thrombosis | | |
| Intravascular | Portal vein thrombosis | US, CT, MRI, Angiography | Hahn P; Mueller P; O'Neill MJ; |
| thrombus | | | Waltman A |
| Intravascular | Pulmonary embolism | CT | McLoud T |
| thrombus | | | |
| Intravascular | Stroke | CT/CTA, MRI/MRA, | Caviness VS; Gonzalez RG; |
| thrombus | | Angiography | Schaefer P; Sorensen AG |
| Ischemic brain tissue | Stroke/ischemic | Diffusion/perfusion, | Gonzalez R; Harris G; |
| | | weighted imaging, MRI, CT | Sorensen AG |
| | | perfusion, CTA | |
| Lumen vascular | Arteriosclerosis/ | CTA, MRA, Angiography | Gonzalez R; Hunter G; Lev M; |
| diameter | peripheral | | Waltman A |
| Macrophage content | Vulnerable plaque | Optical coherence topography | Weissleder R |
| Mean transit time | Stroke | MRI (combined Arterial spin | Harris G; Hoge R; |
| (MTT) | | labeling and Dynamic | Ostergaard L; Rosen B; |
| | | susceptibility contrast), | Sorensen AG; Wald LL |
| | | CAPTIVE | |
| Myocardial blood | Coronary artery | Nuclear medicine, SPECT | Fischman A |
| flow via 13N- | disease | | |
| ammonia | | | |
| Myocardial blood | Ischemic heart | Nuclear medicine, SPECT | Fischman A |
| flow via 13N- | disease | | |
| ammonia | | | |
| Myocardial | Coronary artery | MRI | Brady T; Fischman A |
| contractility/ | disease | Nuclear Medicine | |
| function | | | |
| Myocardial function | Myocarditis | Nuclear medicine | Fischman A |
| contractility | | | |
| Myocardial function | Myocarditis | MRI | Brady T |
| contractility | | | |
| Myocardial perfusion | Coronary artery | Nuclear medicine | Fischman A |
| | disease | | |
| Neuronal activation | Stroke | fMRI, Diffuse optical | Boas D; Mandeville JB; |
| | | topography (Near infrared | Strangman G |
| | | spectroscopy) | |
| Neuronal activation | Stroke | Arterial spin labeling MRI, O2 | Wald LL |
| CMRO2 | | 15 PET, Xenon Enhanced CT | |
| Oxygen consumption | Stroke | Optical imaging | Boas D; Dunn A; Lo E |
| Perfusion/diffusion | Myocarditis | MRI | Brady T |
| Perfusion/diffusion | Myocarditis | Nuclear medicine | Fischman A |
| Perianeurysmal | Inflammatory | CT | Brady T; Saini S; Waltman A |
| fibrosis | abdominal aortic | | |
| | aneurysm (IAAA) | | |
| Splenic tissue | Splenic trauma | MRI | Harisinghani M; Weissleder R |
| characterization | | | |
| Subendothelial lipid | Vulnerable plaque | Optical coherence topography | Brady T; Muller J |
| pool | | Near infrared spectroscopy | |
| Targeted | Vulnerable plaque | Contrast enhanced US | O'Neill MJ; Waltman A |
| mircobubble contrast | | | |
| agents | | | |
| Vascular diameter | Iliac aneurysm | US, CT, MRI | Waltman A |
| and circumference | | | |
| Vascular diameter | Aortic aneurysm | US, CT, MRI | Waltman A |
| and circumference | | | |
| Vascular lumen | Dissection/ | CT, MRI, Angiography | Waltman A |
| diameter | peripheral | | |
| Vascular occlusion | Stroke/ischemic | Diffusion/perfusion, | Gonzalez R; Harris G; |
| | | weighted imaging, MRI, CT | Sorensen AG |
| | | perfusion, CTA | |
| Ventilation/ | Pulmonary embolism | Nuclear medicine | Fischman A |
| perfusion mismatch | | | |

US: Ultrasound;
CT: Computed Tomography;
MRI: Magnetic Resonance Imaging;
CTA: Computed Tomography Angiography;
MRA: Magnetic Resonance Angiography
CAPTIVE: Continuous Assesssment of Perfusion by Tagging Including Volume and water Extraction;
fMRI: Functional Magnetic Resonance Imaging;
SPECT: Single Photon Emission Computed Tomography;
O2 15 PET: Oxygen 15 Positron Emission Tomography

Other examples of data that can be included in the patient-specific heart model include clinical data, such as the results of a six minute walk test. This type of clinical data can be used as a starting point for the computational model by placing the patient into a functional class of heart, for example a New York Heart Association (NYHA) functional class. This can give the clinician a starting point for the computational model which can be used to produce the patient-specific model.

Clinical data can also include data from the patient's medical history, which can be incorporated into the model. For example, if the patient is known to have a mechanical valve, that information can be directly incorporated into the model. Any data that was recorded during surgery can also be fed into the model. Some types of data, such as cardiac output, can be measured during surgery, but are not easily measured externally.

As such, non-DPI parameters of interest include direct, state and observed parameters.

In some embodiments, the initial heart model is modified once to make the patient specific model. In yet other embodiments, the initial model is modified two or more times to produce a dynamic patient specific model. In one embodiment, the system can start with a general computational model that has the properties that are common to all people, and contains many free parameters. As various measurements are obtained, the model is refined, to create a patient-specific heart model. When a measurement is obtained that does not agree with the model, the model can be corrected to account for that new measurement. The model can be continuously refined over time to take into account as many measurements as possible. As such, once the initial patient-specific model is fitted to the available data, the personalized model can be refined over time as new data is obtained. This creates a more robust model that reflects information from a greater number of data points or sources. In a similar manner, the initially produced patient specific heart model can also adjust to changes that may occur in the performance of the patient's heart. As new data comes in that may be different from data previously obtained, or which otherwise conflicts with the model, the model can be adjusted to fit the new data. In patients with an implant, such as a pacemaker, where data is being obtained continuously, this data can be used in the model to continuously adjust the model to match the observed data. This allows the model to be constantly updating to reflect the current performance of the patient's heart.

Following generation of the patient specific heart model, the model may be used in a variety of different further applications. In certain embodiments, the patient specific heart model is employed to gather performance data about the patient's heart and determine an optimal treatment. Specific diagnosis and treatment protocols include, but are not limited, using the patient specific heart model in predicting response to therapy, finding optimal pacing sites for cardiac resynchronization therapy (CRT), etc. For example, for a patient undergoing cardiac resynchronization therapy (CRT), the optimal pacing site can be determined by testing every site in the entire heart, an exhaustive process that is not feasible in the living organ.

The subject methods may be used a variety of different kinds of patients, where the patients are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In certain embodiments, the patients are humans.

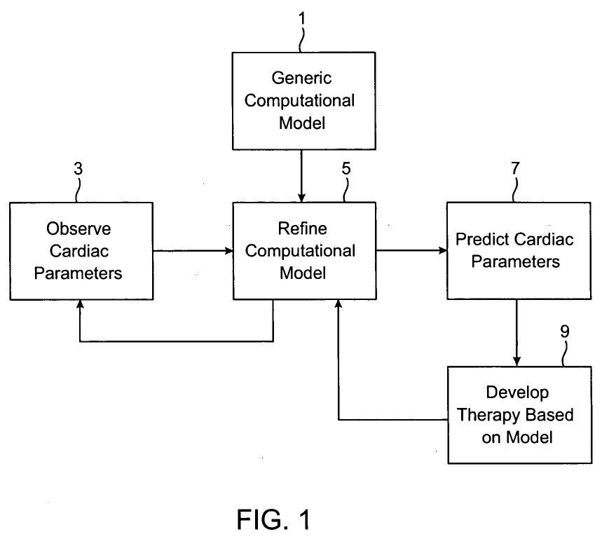

FIG. 1 provides a flow chart of one embodiment of the methods of producing a patient specific heart model. The model starts as a generic computational model 1. One or more patient specific cardiac parameters 3, including ET data, are then obtained. These parameters are then input into the model, e.g., where they can be input directly by the user, or measured by the system itself. Refine computational model block 5 then adjusts the free parameters in the generic model until the results of the model agree with the observed parameters. When new data is available, the system can go back to accepting cardiac parameters block 3, in order to refine the model further. With the patient-specific model at the output of model refinement block 5, the user can virtually measure cardiac parameters and performance parameters under a variety of conditions using predict cardiac parameters block 7. Based on the results of the model predictions, the user can develop a therapy 9. This therapy can be fed back to the refined model to perform the proposed therapy on the model. The model can then be used to predict the resulting cardiac parameters after the therapy is performed. This information can be used to determine whether or not to perform a particular therapy on the patient.

In one embodiment, most of the actions in the blocks of FIG. 1 are performed automatically by software. For example, software can have access to a large library of potential therapies, and perform all of them virtually on the patient specific model. In some embodiments the software can measure several performance parameters, and calculate predicted cardiac performance data, which can include an overall cardiac performance score. The overall cardiac performance score can then be used to suggest to the user the most optimal therapy.

In some embodiments, the therapies can be selected by the user to be performed on the model. The performance parameters on which to base the success of the therapy can also optionally be selected by the user. In another embodiment, the model can take into account the relative risks or side effects of performing each potential therapy when determining which therapy is the optimal one for the patient.

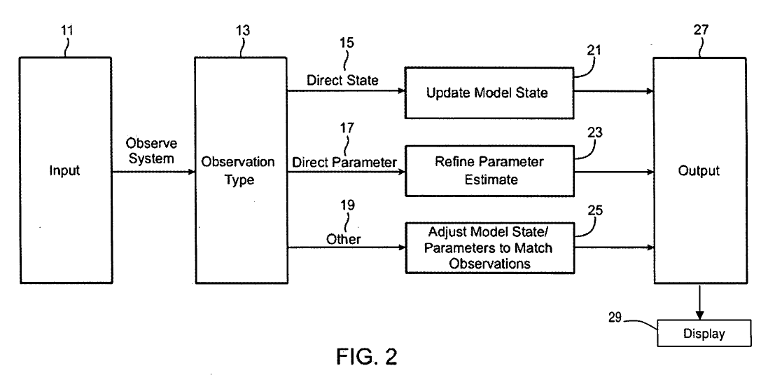

FIG. 2 shows an embodiment of the model refinement process. This can be used in model refinement block 5 from FIG. 1. In some embodiments, the model refinement process can be used to adjust the model based on a variety of data from the patient's heart, until the model most closely resembles the performance of the patient's heart. Input 11 can be observed data from the patient's heart. Block 13 makes the distinction between the different types of data that can be input.

In some embodiments, one or more parameters in addition to the electric tomography data can be used to modify the patient-specific heart model. In this embodiment, observation type block 13 separates the observed data into three general types of data: state parameters or variables 15, direct parameters 17, and other observable parameters 19. State variables 15 are conditions that change from time to time, such as the activity level, drug level (e.g., in the form of a drug dosing schedule (see e.g., a pharmacokinetic model as described in PCT application serial no. PCT/US08/52845 titled “Ingestible Event Marker Systems,” the disclosure of which is herein incorporated by reference) or what sort of heart rhythm the person has. These states can be directly input into the model in block 21 to change the state reflected in the model. For example, if the input data, such as an EKG, suggests that the heart is in atrial fibrillation, the model can be put in atrial fibrillation mode.

Direct parameters 17 are measurable observations that can be directly input into the model. For example, direct parameters can include imaging data or IEGM data. These measurements can be noisy, in which case block 23 can refine the parameter measurement by for example using a common filter or refining the estimate based on multiple parameters or multiple measurements of the same parameter. In some embodiments, the computational or patient-specific heart model can be used to help in the acquisition and/or interpretation of cardiac images. By providing a priori knowledge of the cardiac motion, this can result in improved segmentation of the images, thereby reducing noise and improving accuracy.

There are many other types of data 19 such as indirect observed data, that cannot be directly input into the model, but that can affect other data. For example, the motion of a cardiac lead measured by an ET system can correspond to many different states of the heart and many different sets of parameters. In this case, block 25 can go through an iterative process which adjusts the available parameters in the model until the data predicted by the model converges with the observed data in the patient's heart. The more data which is provided to the model refinement process, the more accurate and robust the model can be.

In any given embodiment, one or more tests can be performed on the patient-specific heart model instead of on the heart itself. This allows the user to perform tests non-invasively, and also allows the user to perform tests that would not be practical in the patient for technological or safety reasons. For example, it is very difficult to measure the total energy consumed by the heart. However, in a predictive computer model, total energy consumption can be computed by integrating at all the nodes over one heart beat and adding them together to give total energy consumption. In this way, observable data can be easily obtained from the model.

The personalized model can also be used to test various treatment options. Many models have a component which can simulate cardiac electrophysiology at the cellular level, which would allow the user to look at the effect of specific drugs. Other treatment options the user could look at include, but are not limited to, the effect of constraint devices, different pacing sites, or a valve replacement. The model can predict the effect of various treatment options on the overall cardiac performance. From this information, the optimum therapy can be chosen, and then performed on the heart itself.

Table 2 shows three possible scenarios where the ET system can be used to test its correlation with the heart model. Based on the information gathered during these tests, the heart model can be modified to fit ET results. This creates a model of the heart which is individualized for the particular subject.

TABLE 2

| | 1.1 Mechanical Function Correlation | 1.2. Electromechanical Function Correlation | 2. Clinical Matrix Simulations |
|---|---|---|---|
| Simulation | Species: Dog | Species: Dog | Species: Human |
| conditions | Physiology: Normal, healthy | Physiology: Normal, healthy | Physiology: Asynchronous dilated heart with |
| and | Model Type: biventricular model with | Model Type: biventricular model with | 1) LBBB 2) LBBB with infarct 3) Natural sinus |
| constraints | closed-loop circulatory system | circulatory system | rhythm 4) AFIB |
| | Model Conditions: Simulated effect | Model Conditions: Simulated effect | Model Type: biventricular model with closed- |
| | of increasing concentrations of | of biventricular pacing. | loop circulatory system |
| | dobutamine (moderate and | RA pacing at 120 beats/min. | Model Conditions: Simulated effect of |
| | high levels) on normal function. | RV pacing on the apical RV | biventricular pacing. |
| | Modulation of LV contractility and | LV pacing on the mid-apical LV free wall | RV pacing on the apical RV |
| | increased dP/dTₘₐₓ | VV delay timing: 1) simultaneous 2) RV first | LV pacing on various positions on the LV free |
| | Animal is RA paced at | by 30 ms. 3) LV first by 30 ms. | wall spanning basal to apical and anterior to |
| | supranormal rate of | | posterior. |
| | 120 beats/min. | | VV delay timing: 1) simultaneous 2) RV first by |
| | | | 30 ms. 3) LV first by 30 ms. |
| Metrics of | LV pressure trace, PV Loop | LV pressure trace, PV Loop | LV pressure trace, PV Loop |
| interest | dP/dT_{max,} Ejection fraction, stroke volume | dP/dT_{max,} Ejection fraction, stroke volume | dP/dT_{max,} Ejection fraction, stroke volume |
| | Total energy expenditure | Total energy expenditure | Total energy expenditure |
| | Material point velocities in reference | Material point velocities in reference | Asynchrony metrics (to be determined) |
| | to the body and with respect to | to the body and with respect | Material point velocities in reference to the |
| | direction of maximal motion. | to direction of maximal motion. | body and with respect to direction of maximal |
| | Four points circling the mitral annulus | Four points circling the mitral annulus | motion. |
| | Two points (basal and apical) on the | Two points (basal and apical) on the | Four points circling the mitral annulus |
| | epicardial LV free wall | epicardial LV free wall | Eight points (basal and apical) on the |
| | Two points in the apical RV (septal wall) | Two points in the apical RV (septal wall) | epicardial LV free wall |
| | | | Two points in the apical RV (septal wall) |
| Analysis | Correlate derived ET velocities to | Correlate derived ET velocities to | Quantify sensitivity and robustness of derived |
| | simulation material point velocities | simulation material point velocities | ET velocity metrics that correlate best with |
| | Confirm morphology of ET velocity | Confirm morphology of ET velocity | cardiac performance metrics |
| | with systolic and diastolic events | with systolic and diastolic events | |
| | Confirm expected cardiac | Confirm expected cardiac | |
| | performance metrics | performance metrics | |

The above methods have been described in terms of patient specific heart models. However, the subject methods can be used to produce a model of the motion or function of non-heart body structures, such as an organ, where in representative embodiments the body structure is an internal body structure, such as an internal organ, e.g., heart, kidney, stomach, lung, etc. Although the invention was described in terms of producing a heart model, the invention is not so limited, the invention being readily adaptable to evaluation of a wide variety of different tissue locations.

### Systems

Also provides are systems that find use in practicing embodiments of the invention. Systems for generating a heart model specific for a patient include a computational heart model, a source of patient-specific dynamic positional information data, and a processor configured to modify the computational heart model with the patient-specific dynamic positional information data to produce a specific model which is predictive for the patient's heart function.

The source for the dynamical positional information data may be a device configured to obtain patient-specific electric tomography data, or may be a device that contains information on a patient's dynamic positional information data, e.g. a program or processor.

In some embodiments, the processor is configured to modify the patient-specific heart model with one or more additional parameters. The parameters can include state parameters, direct parameters, or observed parameters. In some embodiments, the system is configured to perform one or more tests on the patient-specific heart model. In some embodiments, the system is configured to generate predicted cardiac performance data specific for a patient, including an overall cardiac performance score. The results of the tests and/or tested therapies can be used for both diagnosis and treatment. In some embodiments, the testing can include testing for an optimal pacing site for patients undergoing cardiac resynchronization therapy (CRT),

In some embodiments, the system includes a visual representation of the patient-specific heart model. In some embodiments, a display 29 in FIG. 2 provides a visual representation of the model. The displayed data may be displayed in any convenient format, e.g., provided on a display of a computer monitor, printed onto a substrate, such as paper, etc. The displays may be in the form of plots, graphs, or any other convenient format, where the formats may be two dimensional, three-dimensional, and include data from non-heart model sources, etc. In some embodiments the display can employ color-mapping, etc. of various parameters in any manner than increases the ability of the observer or clinician to interpret the data. Quantitative data regarding the heart model may also be presented on the display. For example, in one embodiment, the patient-specific model can drive a visual representation of the beating heart, which can be displayed on a computer screen or other visual display. In some embodiments, the visual display can also include data from other sources (e.g., imaging data, EPS data, EKG data, real-time data on catheter location) which can be displayed simultaneously or sequentially. Displays of interest include, but are not limited to: those disclosed in U.S. patent application Ser. No. 11/731,786 titled “Electric Tomography” and filed on Mar. 30, 2007, the disclosure of which is herein incorporated by reference.

In other embodiments, the system can include a graphical user interface (GUI) for data display. The phrase “graphical user interface” (GUI) is used to refer to a software interface designed to standardize and simplify the use of computer programs, as by using a mouse to manipulate text and images on a display screen featuring icons, windows, and menus. GUIs of interest include, but are not limited to: those disclosed in U.S. application Ser. No. 11/909,786, the disclosure of which is herein incorporated by reference. GUI displays can be tailored to assist the clinician during clinical situations, such as testing of potential pacing sites for CRT, and can be used to provide the user with ways to test various treatments and gather quantitative data from the model.

### Utility

The present invention provides the clinician an important new tool in their therapeutic armamentarium: a patient-specific model of the heart that is predictive of the performance of the patient's heart under a variety of conditions. By performing tests on the heart model, the user, such as a physician, can obtain cardiac data which is not easily obtained in the patient's heart itself. Applications include, but are not limited to: (1) diagnosis and treatment of cardiac conditions; (2) predicting response to therapy based on tests of the patient-specific model; (3) generating a cardiac performance index for a baseline condition or a particular therapy; and (4) testing the effect of pacing in the heart at multiple sites to find the optimal site for CRT.

For example, the ET constrained heart model can be very useful in determining the optimal pacing site for cardiac resynchronization therapy. Consider a patient who is to undergo a pacemaker implant, or a patient with an existing CRT implant who is a non-responder possibly due to poor lead placement. Given a model of the heart that corresponded well to the patient's heart, the user can try pacing at every site in the heart model to determine the optimal pacing site. The user could choose to define the optimum site in terms of cardiac output, ejection fraction, cardiac efficiency or other parameters that can be determined in the model. These performance parameters are often difficult to measure in the patient itself. With the present invention, the process of finding the optimum site is capable of being substantially, if not completely, automated. The process could be based on a single performance parameter, or based on some combination of two or more parameters.

When the optimum site for pacing is determined, an interventional procedure can be used that may be less invasive than the standard procedure, since the exact destination of the lead is known, and multiple sites do not need to be tested in vivo. For example, if the optimum site were found to be along a vein, a transvenous intervention can be chosen that would traverse that vein. Alternatively, for other locations that are not along a vein, an epicardial intervention can be performed to go directly to the correct location through a small incision. Because the surgeon would know before the surgery the exact destination of the lead for optimum performance, the implant procedure can be shorter in duration, less invasive, and ultimately safer and more effective for the patient.

Similarly, in a case of a patient with an arrhythmia or ectopic focus, where ablation is desired, the present invention makes it possible to test the effects of various ablation locations on the patient-specific heart model, thereby saving time and reducing radiation exposure during the actual procedure, because the optimal site for therapy can be determined in advance.

Another case where the constrained heart model can be beneficial is in patients with congenital heart defects. These patients are often candidates for surgeries that can be very risky. With a model that corresponds accurately to the patient's individual heart, the surgery can be performed virtually on the model, and the results can be used to determine whether or not to perform the surgery on the patient.

Additionally, with a large number of patients for which a patient-specific computational model has been produced, sub-groups can be generated that have similar characteristics. For example, all patients with a mechanical heart valve, or all patients in atrial fibrillation can be grouped together. This can lead to improved categorization of a patient's cardiac function based on a preliminary set of data, which can then be used to determine a proper treatment.

In some embodiments, the patient-specific model or a sub-group specific model can be used to simulate various cardiac pathologies such as an arrhythmia, an ectopic focus, bundle branch block, atrial or ventricular fibrillation, radio-frequency ablation, areas of infarcted myocardium, aneurysms, or scars, etc. Various tests and potential therapies can then be tested on the patient-specific model or sub-group specific model in order to find an optimal treatment or treatment strategy.

In the case of a patient undergoing cardiac resynchronization therapy (CRT), in which one or more leads are implanted in the heart and controlled by a pacemaker to restore normal cardiac rhythm, the constrained patient-specific heart model can be especially useful. A significant number of CRT patients are non-responders, meaning they do not benefit significantly from CRT. Often, this is due to poor lead placement, in which the pacing electrodes do not properly capture the heart tissue. Using the constrained heart model, the user can test the effect of pacing at every site on the heart in order to find the optimal site. Once this site is chosen, it can be reached much less invasively, since the surgeon will know the exact destination for the cardiac lead.

Non-cardiac applications will be readily apparent to the skilled artisan, such as, by example, evaluating and predicting the effects of therapy on distention of the urinary bladder, congestion in the lungs or fluid in the lower extremities, the amount of fluid in the cerebral ventricles and other fluid spaces in the brain and spinal cord, etc. Other applications can also include assessing variable characteristics of many organs of the body such as the stomach, vascular system, or any organ or system in the body in which a computational model can be provided, which can subsequently be modified by dynamic positional information data to produce a more robust and complex model. These patient-specific models can then be used to predict the results of any intervention or therapy, thereby generating data which may be impossible to obtain, or more difficult to obtain, in the living subject.

### Computer Readable Storage Medium

One or more aspects of the subject invention may be in the form of computer readable storage media having a processing program stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

Computer readable storage media may include a stored processing program, which operates a processor to operate a system and methods of the subject invention. The computer readable storage medium may include programming embodying an algorithm for carrying out the subject methods. Accordingly, such a stored algorithm is configured to, or is otherwise capable of, practicing the subject methods, e.g., by modifying a computational model of the heart to produce a patient-specific heart model, or by obtaining dynamic positional information data from a patient. The subject algorithm and associated processor may also be capable of implementing the appropriate adjustment(s). In certain embodiments, systems loaded with such computer readable storage mediums such that the systems are configured to practice the subject methods are provided.

### Kits

Also provided are kits for practicing the subject methods. Kits may include programming configured to modify a computational heart model with patient-specific dynamic positional information data to generate a specific heart model, as described above. The kits may include programming configured to modify a heart model with additional non-DPI patient-specific parameters, such as the geometry of the heart, the contractility in various places, the vascularization, properties of the conductive bundles, the fluid dynamics of the blood, etc. The kits may also include programming configured to modify a heart model with data generated from the tests or therapies performed on the patient. The programming can also include generating an overall cardiac performance score.

In some embodiments, the subject kits can include programming to produce a visual representation of the specific heart model. In some embodiments the kit can also include a graphical user interface (GUI), and/or a computer readable storage medium having the processing program stored thereon.

The subject kits may also include instructions for how to practice the subject methods using the components of the kit. The instructions may be recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for producing a patient specific heart model that is specific for a patient, said method comprising:
(a) providing a computational model of a heart;
(b) obtaining dynamic positional information (DPI) data from said patient; and
(c) modifying said computational model with said DPI data to produce said patient specific heart model, wherein said patient specific heart model is predictive for said patient's heart function.

2. The method according to claim 1, wherein said modifying further employs a non-DPI patient specific parameter.

3. The method according to claim 2, wherein said non-DPI patient specific parameter is a state parameter.

4. The method according to claim 3, wherein said state parameter is a drug dosing parameter.

5. The method according to claim 2, wherein said non-DPI patient specific parameter is a direct parameter.

6. The method according to claim 2, wherein said non-DPI patient specific parameter is an observed parameter.

7. The method according to claim 1, wherein said DPI data is electrical tomography data.

8. The method according to claim 1, wherein said method further comprises performing a test on said specific heart model.

9. The method according to claim 8, wherein said test comprises determining an optimal pacing site.

10. The method according to claim 8, wherein said test generates predicted cardiac performance data specific for said subject.

11. The method according to claim 10, wherein said predicted cardiac performance data is a cardiac performance score.

12. The method according to claim 10, wherein said method further comprises employing said predicted cardiac performance data in the diagnosis or treatment said patient.

13. The method according to claim 12, wherein said treatment is cardiac resynchronization therapy (CRT).

14. The method according to claim 4, wherein said direct parameter is selected from the group consisting of: imaging data, angiographic data, EPS data, and IEGM data.

15. The method according to claim 2, wherein said parameter is selected from the group consisting of:
electrical delay time, data from pressure-volume catheters, anatomic disruption of arterial wall, aneurysmal wall thickness, atherosclerotic plaque temperature, cerebral blood flow, cerebral blood volume, CMRO2, fibrous cap thickness, flow heterogeneity, increased pulmonary interstitial markings, intraluminal hemorrhage, intravascular thrombus, ischemic brain tissue, macrophage content, mean transit time, myocardial blood flow via 13N-ammonia, myocardial contractility/function, myocardial perfusion, neuronal activation, neuronal activation CMRO2, oxygen consumption, perfusion/diffusion, perianeurysmal fibrosis, splenic tissue characterization, subendothelial lipid pool, targeted microbubble contrast agents, vascular diameter and circumference, vascular lumen diameter, vascular occlusion, ventilation/perfusion mismatch, fibrilation state, activity state, heart rate, heart rate variability, fluid status and respiration rate.

16. A system for generating a patient specific heart model that is specific for a patient, said system comprising:
(a) a computational heart model;
(b) a source of patient-specific dynamic positional information (DPI) data; and
(c) a processor configured to modify said computational heart model with patient-specific DPI data to produce said a patient specific heart model.

17. The system according to claim 16, wherein said source is a device configured to obtain patient-specific DPI data.

18. The system according to claim 16, wherein said source is a device containing patient-specific DPI data.

19. The method according to claim 16, wherein said processor is configured to modify said patient-specific heart model with a non-DPI patient specific parameter.

20. The system according to claim 16, wherein said system is configured to perform a test on said specific heart model.

21. The system according to claim 20, wherein said test comprises determining an optimal pacing site.

22. The system according to claim 20, wherein said test generates predicted cardiac performance data specific for said patient.

23. The system according to claim 22, wherein said predicted cardiac performance data is an a cardiac performance score.

24. The system according to claim 22, wherein said system is configured to employ said predicted cardiac performance data is in the diagnosis or treatment of said patient.

25. The system according to claim 24, wherein said treatment is cardiac resynchronization therapy (CRT).

26. The system according to claim 16, wherein said system further includes a visual representation of said patient specific heart model.

27. A computer readable storage medium having a processing program stored thereon, wherein said processing program operates a processor to operate a system to perform a method comprising:
(a) providing a computational model of a heart;
(b) obtaining dynamic positional information (DPI) data from said patient; and
(c) modifying said computational model with said DPI data to produce said patient specific heart model, wherein said patient specific heart model is predictive for said patient's heart function.

28. A kit comprising programming configured to modify a computational heart model with patient-specific DPI data to generate a specific heart model in a method comprising:
(a) providing a computational model of a heart;
(b) obtaining dynamic positional information (DPI) data from said patient; and
(c) modifying said computational model with said DPI data to produce said patient specific heart model, wherein said patient specific heart model is predictive for said patient's heart function.
